(19) United States
(12) Patent Application Publication
(10) Pub. No.: US 2016/0066797 A1
(43) Pub. Date: Mar. 10, 2016

(54) COMPOUND MEDICAL DEVICE

(71) Applicants: Byung-Hoon LEE, Seoul (KR); SNU R&DB FOUNDATION, Seoul (KR)

(72) Inventors: Byung-Hoon LEE, Seoul (KR); Ju Han KIM, Seoul (KR)

(21) Appl. No.: 14/781,555

(22) PCT Filed: May 21, 2014
(86) PCT No.: PCT/KR2014/004514

§ 371 Date: Sep. 30, 2015

(30) Foreign Application Priority Data
May 22, 2013 (KR) 10-2013-0057735

(51) Int. Cl. A61B 5/0205; A61B 7/04; A61B 1/06; A61B 7/00; A61B 8/14; A61B 1/227; A61B 5/01; A61B 5/00

(52) U.S. Cl. CPC A61B 5/02055; A61B 5/01; A61B 7/045; A61B 5/7282; A61B 7/003; A61B 7/008; A61B 5/4343; A61B 8/14; A61B 1/227; A61B 1/0661; A61B 2503/06; A61B 2503/02; A61B 5/02411

(57) ABSTRACT

A compound medical device, which accurately estimates the disease state of a subject or determines the gestational week or health status of a subject, may include an auscultation unit, receiving bodily sound, a body temperature measurement unit, measuring the body temperature of a subject, a controller unit, connected to the auscultation unit and the body temperature measurement unit and estimating the disease state or determining the gestational week or health status of the subject based on the input received from the auscultation unit and the body temperature measurement unit, and an output unit, outputting the disease state or the gestational week or health status of the subject estimated or determined by the controller unit.

### TECHNICAL FIELD

The present invention is related to a compound medical device, and more particularly, to a compound medical device that may diagnose a disease or health status of a patient in consideration of auscultation sounds and a body temperature at the same time.

### BACKGROUND ART

When a doctor examines a patient in a hospital, the doctor makes a first diagnosis by listening to a history of disease of the patient and performing inspection by observing the patient, palpation by touching the patient with hands, percussion by tapping the patient, auscultation by using a stethoscope, and using a blood pressure measuring device that detects blood pressure variation. Based on the first diagnosis, the doctor performs a comprehensive examination and makes a precise diagnosis of a disease.

Recently, when performing auscultation by listening to auscultation sounds with a stethoscope and diagnosing based on the sounds, instead of diagnosing based on auscultation sounds that are directly heard by the doctor, the doctor may use electronic stethoscopes that can issue a diagnose by electrically analyzing auscultation sounds detected via a stethoscope.

In the electronic stethoscopes, a processor electrically analyzes types of auscultation sounds that vary according to auscultated portions so that an objective auscultation result is provided.

Examples of such electronic stethoscopes include patented inventions and utility models that are devised by the applicant of the present patent application.

For example, Korean Utility Model Registration No. 20-0131518 (registered on Sep. 16, 1998) and U.S. Pat. No. 5,737,429 (registered on Apr. 7, 1998) disclose a portable viewable stethoscope, which is invented by the applicant and has a simple structure without rubber sound tubes and earpieces. A case of the stethoscope houses large and small sound absorbing cups for collecting sounds, an electronic amplifying circuit, a speaker, and an image display. Accordingly, accumulated auscultation sounds may be amplified to be audible, and simultaneously, auscultation frequency waveforms are displayed as a graph on the image display to be observed by people at the same time.

Also, Korean Patent No. 10-0387201 (registered on May 29, 2003) and U.S. Pat. No. 6,520,924 (registered on Feb. 18, 2003) disclose an automatic diagnostic apparatus, which is invented by the applicant and filters noise coming from outside the body from sounds auscultated by an auscultation microphone, converts filtered analog waveform data into digital data by using an analog/digital (A/D) converter, finds out names of diseases by searching and comparing standard auscultation sounds of various diseases, and simultaneously, records the names of diseases to a recording device and displays the names of diseases on a plurality of monitors to be viewable by people.

Also, Korean Utility Model Registration No. 20-0384551 (registered on May 9, 2005) and U.S. Pat. No. 8,200,277, disclose a mobile phone with a stethoscope, which is invented by the applicant and may use a transmission and reception function of the mobile phone to transmit and receive data that is auscultated by a stethoscope so as to provide a telemedicine service.

Also, Korean Patent Registration No. 10-1178867 (registered on Aug. 27, 2012) discloses a telemedical stethoscope, which is invented by the applicant and automatically diagnoses a disease, and visually and auditorily records and stores auscultation data on a screen.

Infectious diseases may be classified as local infection and systemic infection. An infection is a reaction of the body to a disease, and may cause three signs such as local fever, redness, and swelling.

For example, signs and symptoms of the systemic infection include pains, chills, fevers (increase in body temperature), nauseous (dizziness, or queasiness with an impulse to vomit), and vomiting. Symptoms of the local infection include whole body fever, local fever, local pain, redness and swelling at the affected side, and pus and pus smell.

When diagnosing with only auscultation sounds, fever, which is one of the symptoms of infectious diseases, cannot be effectively detected, and thus it is difficult to accurately diagnose the infectious diseases. Also, it is difficult to accurately diagnose the cause of fever by only measuring a body temperature.

### DETAILED DESCRIPTION OF THE INVENTION

### Technical Problem

The present invention is devised with regard to the description above, and provides a compound medical device that may accurately determine symptoms (in particular, symptoms of infectious diseases) of a patient by performing a first diagnosis with regard to the patient by applying algorithms that utilize all pieces of data accumulated through an electrical analysis of auscultation sounds and body temperature measurement.

### Technical Solution

According to an embodiment of the present invention for solving the problem above, a compound medical device includes an auscultation unit receiving sounds inside a body of a patient, a body temperature measurement unit measuring a body temperature of a contact portion, a controller connected to the auscultation unit and the body temperature measurement unit, and determining health status of the patient based on an input received from the auscultation unit and the body temperature measurement unit, and an output unit outputting the health status of a patient determined by the controller.

According to another embodiment of the present invention, the compound medical device further includes a body data input unit receiving at least one of age, sex, height, weight, gestational week, and a mode of diagnosis of the patient from a user.

According to another embodiment of the present invention, when the mode of diagnosis is a pneumonia mode, the patient is diagnosed with pneumonia by the controller when both of a pneumonia diagnosis body temperature criterion and at least one of pneumonia diagnosis auscultation criteria are satisfied, the pneumonia diagnosis auscultation criteria include a first pneumonia diagnosis auscultation criterion, a second pneumonia diagnosis auscultation criterion, and a third pneumonia diagnosis auscultation criterion, the first pneumonia diagnosis auscultation criterion is met when the auscultation unit detects rales, crackles, or moist rales, the second pneumonia diagnosis auscultation criterion is met when the auscultation unit fails to detect respiration rate during a pulmonary region auscultation, and the pneumonia diagnosis body temperature criterion is met when the body temperature measurement unit detects fever that exceeds a predetermined range.

According to another embodiment of the present invention, when the mode of diagnosis is an enteritis mode, the patient is diagnosed with enteritis by the controller when a first enteritis diagnosis auscultation criterion and a second enteritis diagnosis auscultation criterion are satisfied, the first enteritis diagnosis auscultation criterion is met when the auscultation unit detects metallic bowel sound, and the second enteritis diagnosis auscultation criterion is met when a heart rate detected by the auscultation unit exceeds a statistically normal range and a body temperature increase rate detected by the body temperature measurement unit exceeds a statistically normal range.

According to another embodiment of the present invention, when the mode of diagnosis is a prenatal checkup mode, the patient is diagnosed with a normal pregnancy by the controller when both of a pregnancy heart rate criterion and a pregnancy body temperature criterion are satisfied, the pregnancy heart rate criterion is met when a fetal heart rate detected by the auscultation unit is within a predetermined range according to a normal fetal heart rate chart, and the pregnancy body temperature criterion is met a body temperature of the patient detected by the body temperature measurement unit is within a predetermined range according to a normal basal body temperature chart.

### Advantageous Effects of the Invention

A compound medical device according to the present invention may accurately diagnose diseases with fever by diagnosing a patient by using algorithms that may examine health status of the patient in consideration of a body temperature and auscultation sounds at the same time.

Also, the compound medical device according to the present invention may be capable of performing objective diagnosis by electrically processing the body temperature and auscultation sounds and diagnosing a disease according to algorithms.

Also, the compound medical device according to the present invention may examine health status of a pregnant woman at each week of a pregnant period by using the body temperature and auscultation sounds, affecting a fetus relatively less than an ultrasound unit that examines cardiac sounds by using ultrasound.

### DESCRIPTION OF THE DRAWINGS

FIG. 1 is a perspective view of a compound medical device according to an embodiment of the present invention.

FIG. 1A is a perspective view of a compound medical device according to another embodiment of the present invention.

FIG. 1B is a perspective view of a compound medical device according to another embodiment of the present invention.

FIG. 1C is a perspective view of a compound medical device according to another embodiment of the present invention.

FIG. 1D is a perspective view of a compound medical device according to another embodiment of the present invention.

FIG. 2 is a cross-sectional view of the compound medical device of FIG. 1.

FIG. 3 is a block diagram of a central processing module in a stethoscope of the present invention.

### EMBODIMENTS

Various embodiments will be described with reference to the accompanying drawings. Like reference numerals in the drawings denote like elements. Various descriptions are provided to facilitate understanding of the present invention.

However, it is evident that such embodiments may be implemented without detailed description. In other examples, well-known structures and apparatuses are provided in block diagrams for convenient description of the embodiments.

In the present specification, the terms “component,” “module,” and “system” refer to computer-related entity, hardware, firmware, software, a combination of software and hardware, or execution of software. For example, a component may be, but is not limited to, processes executed in a processor, a processor, an object, an execution thread, a program, and/or a computer. For example, an application executed in a computing device and a computing device may both be components. One or more components may be included in a processor and/or an execution thread, and a component may be localized in one computer, or distributed among two or more computers. Also, such components may be executed from various computer-readable media that include various data structures. For example, the components may communicate via local and/or remote processing according to signals (e.g., data from a component that interacts with another component in a local system or a distribution system, and/or data from another system and a network such as the Internet transmitted through signals) with at least one data packet.

The descriptions of the embodiments are provided such that one of ordinary skill in the art to which the present invention pertains may use or implement the present invention. Various modifications of the embodiments will be evident to one of ordinary skill in the art to which the present invention pertains. General principles defined herein may be applied to other embodiments without departing from the scope of the present invention.

Therefore, the present invention is not limited to the embodiments provided herein, but should be interpreted in a broad sense that is consistent to principles and new features described herein.

A compound medical device of the present invention includes a case in which a sound absorbing cup including an auscultation microphone is integrally formed with the case by a supporting tube. A control module and a power source are included in the case. A flat liquid crystal display (LCD) window for visually displaying data is provided at an upper side of the case. A switch block including a power switch, a visual and auditory switch, and a remote transmission and reception switch is provided at a side of the case.

The LCD window may be configured as a touch screen including a touch panel. When the LCD window is configured as a touch screen including a touch panel, a user may input at least one of age, sex, height, weight, gestational week, and mode of diagnosis.

The switch block may include a number pad or a keypad. When the switch block includes a number pad or a keypad, the user may input at least one of age, sex, height, weight, gestational week, and mode of diagnosis by using the number pad or the keypad.

The touch screen including a touch panel and the switch block including a keypad or a number pad may operate as a user data input unit.

Also, a mode switch block may be included at an upper side of the case. The mode switch block may include a designated switch for a heart region auscultation mode, a designated switch for a pulmonary region auscultation mode, a designated switch for a pregnant woman health status auscultation mode, and a designated switch for a bowel region auscultation mode. The mode switch block may be incorporated with the user data input unit. In this case, a mode switch may be omitted.

Also, a speaker for auditorily listening data and an antenna for remotely transceiving data may be provided at another side of the case and electrically connected with each other.

A contact unit of the sound absorbing cup, which directly contacts the body of a patient, may include a temperature sensor. The temperature sensor may be formed of an element of which resistance changes according to contact, and thus, a body temperature of the patient may be electrically detected by a change in a value of current flowing in the temperature sensor or in a value of a voltage drop occurring in the temperature sensor, according to a change in the body temperature of the patient.

As described above, the temperature sensor may be formed as a contact temperature sensor. Alternatively, the temperature sensor may be formed as a non-contact temperature sensor. When the temperature sensor is formed as a non-contact temperature sensor, the temperature sensor does not have to be located at an area of the sound absorbing cup which contacts the body, but may be located at various areas in the sound absorbing cup.

Alternatively, the temperature sensor and the sound absorbing cup may be provided separately, and the temperature sensor may be electrically connected to the compound medical device.

The control module may include an auscultation mode designation unit designating an auscultation mode by using the mode designated switch, a detector including an auscultation microphone, a filter filtering only auscultation sounds of a designation mode from the detected auscultation sounds and removing sounds of other body parts or noise outside the body, an auscultation signal amplifier amplifying filtered auscultation signals, an analog/digital (A/D) converter converting amplified analog waveform signals into digital signals, and an amplifier for the digital signals. The auscultation mode designation unit, the detector, the filter, the auscultation signal amplifier, the A/D converter, and the amplifier may form an auscultation unit. The auscultation sounds may include blood flow sounds, breathing sounds, and gastro-intestinal movement sounds. When the auscultation sounds includes blood flow sounds, breathing sounds, and gastro-intestinal movement sounds, a controller may use an algorithm for analyzing each of the auscultation sounds.

Also, the auscultation unit may include an ultrasound transmitter generating ultrasound waves and transmitting the ultrasound waves into the body of the patient, and an ultrasound receiver receiving echoes of the ultrasound waves transmitted into the body of the patient.

When the auscultation unit includes the ultrasound transmitter or the ultrasound receiver, the controller may analyze the received echoes of the ultrasound waves and reproduce the echoes as a 2-dimensional or 3-dimensional image.

The reproduced image may be displayed via an output unit of the compound medical device, or transmitted to the outside (e.g., a medical data server, another medical device, a mobile device such as a smartphone, or a server providing information to the mobile device) of the compound medical device by a transceiver.

Also, the control module may include a body temperature measurement unit that is connected to the temperature sensor and measures the body temperature of the patient.

Also, the control module may include a database that stores standard data of various diseases or a pregnant woman body temperature-heart rate table for each mode, a comparing unit searching for and comparing detected digital data with standard data of a designated mode selected in the database, the controller storing various execution programs for reading found names of diseases and controlling each unit, a storage storing read information, an output unit outputting the read information, an LCD unit visually displaying data output from the output unit on the LCD window, an audio unit auditorily outputting the output data to the speaker to be audible, and a transceiver transmitting and receiving output data to and from a remote medical center and other medical institutions via the antenna.

The transceiver may transmit, to the outside, information that is read by the controller and/or medical data of the patient which is detected by the auscultation unit or the body temperature measurement unit. For example, the transceiver may transmit the above-described data to a medical data server, another medical device, a mobile device such as a smartphone, or a server providing information to the mobile device. The transceiver may transmit body temperature data, auscultation sounds data, and ultrasound echo data of the patient which are detected by the body temperature measurement unit and the auscultation unit to the outside, or transmit a diagnosis result obtained by analyzing the above-described data to the outside. When the diagnosis result is not transmitted to the outside and only the body temperature data, the auscultation sounds data, and the ultrasound echo data of the patient are transmitted to the outside, the compound medical device does not perform a diagnosis function by using the above-described data, but may simply perform a function of transmitting the data to an external diagnosis device. The external diagnosis device may include a medical data server, another medical device, a mobile device such as a smartphone, or a server providing information to the mobile device.

For example, the compound medical device may transmit the body temperature data, the auscultation sounds data, and the ultrasound echo data of the patient to a smartphone. The smartphone may determine health status (a name of disease, pregnancy status, etc.) of the patient based on the received data. Alternatively, the smartphone may transmit the received data to a server that may be accessed by the smartphone, and may output health status of the patient that is determined by the server.

The transceiver may include a transmission chain and/or a reception chain that may perform wireless transmission and wireless reception with an external device. Alternatively, the transceiver may be a wired interface that may be wired to the external device.

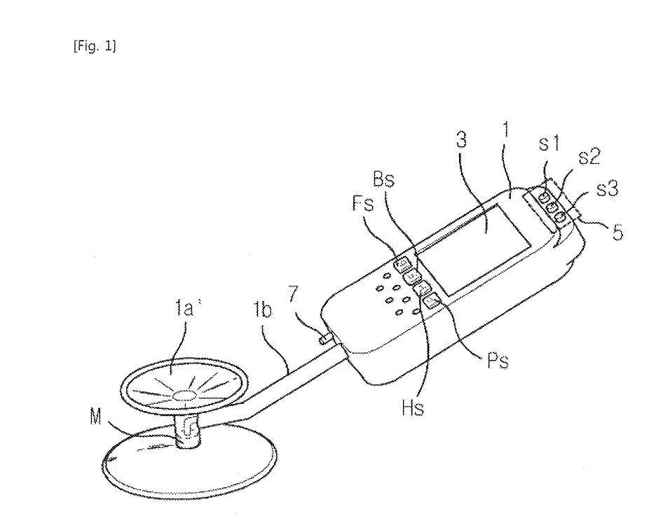

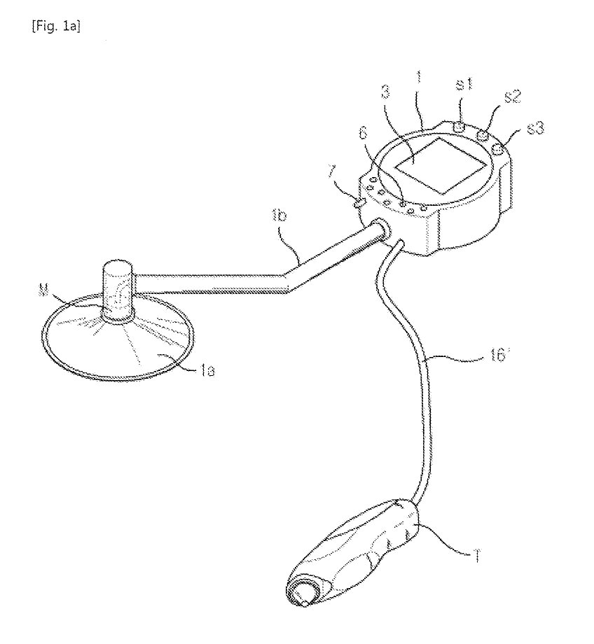

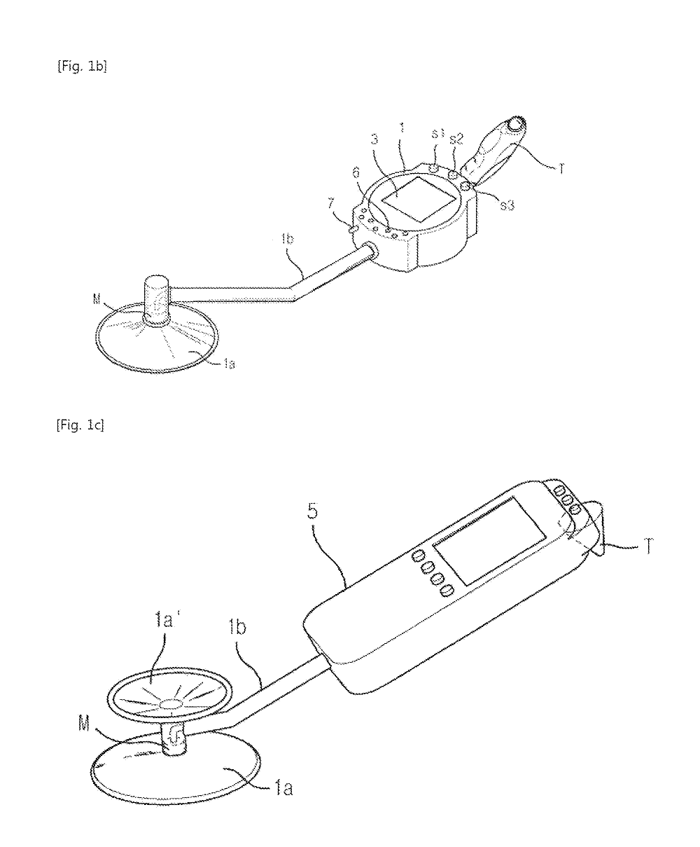

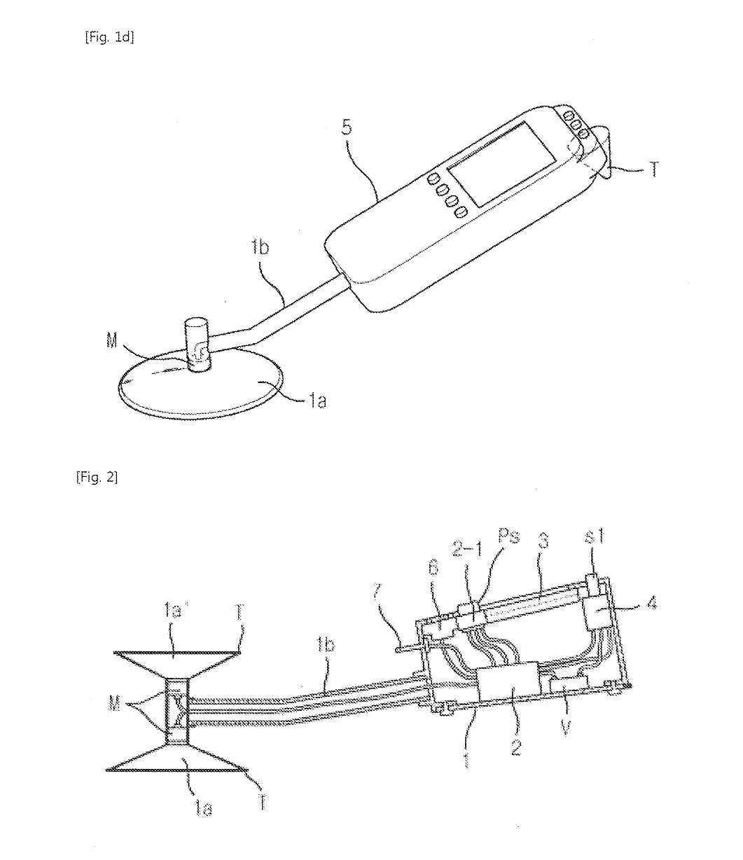

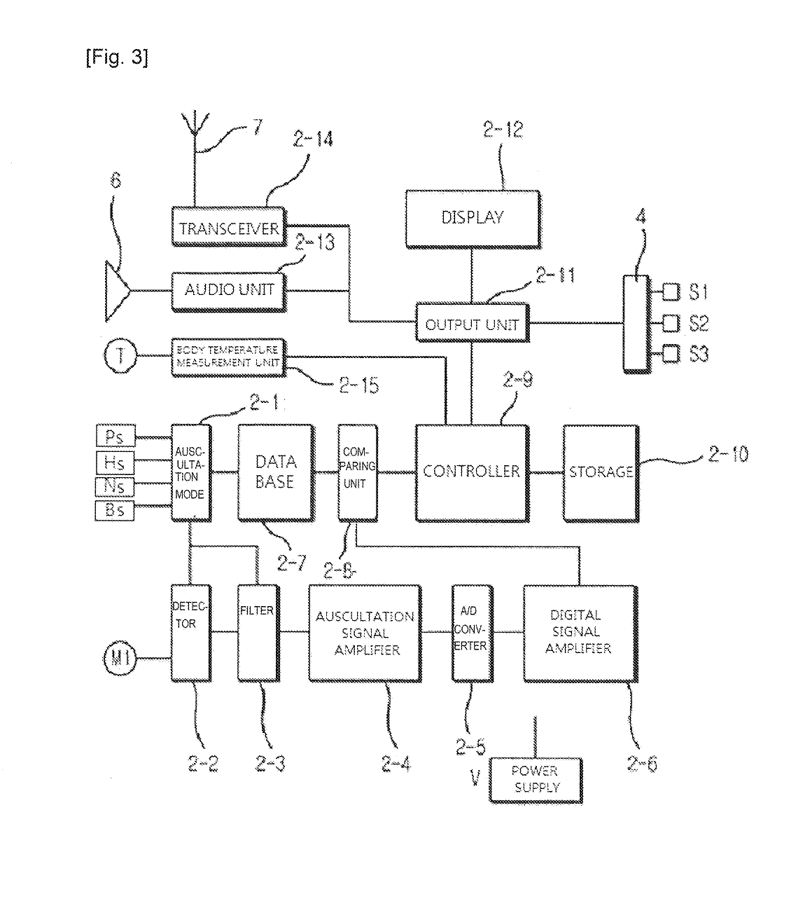

As shown in FIGS. 1 and 2, one or more sound absorbing cups 1a and 1a′ including an auscultation microphone M are integrally formed with a case 1 by a supporting tube 1b. A central control module 2 and a power supply (battery) V are mounted in the case 1. An LCD window 3 is provided at an upper portion of the case 1. At another side of the case 1, a switch block 4 including a power switch s1, a view switch s2, and a remote transmission and reception switch s3 is provided. At another side of the case 1, a mode switch block 5 including a designated switch Hs for a heart region auscultation mode H, a designated switch Ps of a pulmonary region auscultation mode P, a designated switch Fs of a pregnant woman health status auscultation mode F, and a designated switch Bs of a bowel region auscultation mode B, a speaker 6 for listening to data, and an antenna 7 that remotely transmits and receives data.

However, as described above, auscultation mode switches included in the switch block 4 are only exemplary. The above-described switches may be replaced by a separate input device such as a touch screen.

Also, a temperature sensor T may be provided along a portion of the one or more sound absorbing cups 1a and 1a′ including the auscultation microphone M which contacts a patient. The temperature sensor T may be electrically connected with the central control module 2 via the supporting tube 1b, and be used to measure the body temperature of the patient.

FIG. 1A illustrates an embodiment in which the temperature sensor T is not attached to a sound absorbing cup but provided on a separate thermometer.

The temperature sensor T may be connected with the case 1 via a separate supporting tube 1b′ that is different from the supporting tube 1b that is connected to the microphone M. The supporting tube 1b′ may be formed of an elastic material so that the temperature sensor T may be freely moved.

The temperature sensor T may be a contact temperature sensor or a non-contact temperature sensor that measures the body temperature by using infrared rays. When the temperature sensor T is a non-contact temperature sensor, the temperature sensor T may emit infrared rays onto the body of a patient, detect wavelengths of reflected infrared rays, and thus measure the body temperature of the patient.

Additionally, a portion of the thermometer where the temperature sensor is provided may include a lighting unit (not shown) and a magnifying lens (not shown) that may illuminate an inside of the body of the patient. The lighting unit that may illuminate an inside of the body of the patient and the magnifying lens may include an otoscope.

FIG. 1B illustrates an embodiment in which the temperature sensor T is not connected with the case 1 via a supporting tube, but a temperature sensor is directly connected with the case 1 of the compound medical device. As described above, the embodiment in which the temperature sensor is directly connected with the case 1 of the compound medical device may be indicated as a thermometer embedded type stethoscope.

FIGS. 1C and 1D illustrate other embodiments in which the temperature sensor T is directly connected to the case 1.

As shown in FIG. 3, the central control module 2 of the compound medical device may include an auscultation mode designation unit 2-1 designating an auscultation mode, a detector 2-2 to which the auscultation microphone M is connected, a filter 2-3 filtering only auscultation sounds of a designated mode from detected auscultation sounds and removing sounds of other body parts or noise outside the body, an auscultation signal amplifier 2-4 amplifying filtered auscultation signals, an A/D converter 2-5 converting amplified analog waveform signals into digital signals, a digital signal amplifier 2-6, a database 2-7 storing standard data of various diseases according to auscultation modes, a comparing unit 2-8 comparing detected digital data with standard data of a designated mode that is stored in the database 2-7, a central controller 2-9 reading found names of diseases and controlling each unit, a storage 2-10 of read information, an output unit 2-11 outputting the read information, a display 2-12 visually displaying data output from the output unit 2-11 on the LCD window 3, an audio unit 2-13 auditorily outputting the output data to the speaker 6 for listening, a transceiver 2-14 transmitting and receiving the output data to and from a central medical center or a remote medical institution via the antenna 7, and a body temperature measurement unit 2-15 connected to the temperature sensor T and measuring the body temperature of the patient.

A method performed by the compound medical device of the present invention of measuring and analyzing auscultation sounds according to auscultated portions by using auscultation modes refers to the Korean Patent Application No. 10-1178867 of the Applicant of the present application, filed on Mar. 30, 2011 and registered on Aug. 27, 2012.

Hereinafter, an algorithm for diagnosing pneumonia infection by using auscultation sounds and body temperatures in a pulmonary auscultation mode will be described.

When a mode of diagnosis is a pneumonia mode, a patient may be diagnosed with pneumonia by the controller of the compound medical device when a pneumonia diagnosis body temperature criterion and at least one of pneumonia diagnosis auscultation criteria are both satisfied.

The pneumonia diagnosis auscultation criteria include a group formed of a pediatric pneumonia diagnosis criterion, a first pneumonia diagnosis auscultation criterion, and a second pneumonia diagnosis auscultation criterion.

The pediatric pneumonia diagnosis criterion may be applied when the patient is a child. The pediatric pneumonia diagnosis criterion may be met 60 or more breaths per minute in children under 2 months old, 50 or more breaths per minute when the patient is a child between 2 to 11 months old, or 40 times or more breaths per minute when the patient is a child between 11 to 59 months old.

Also, the first pneumonia diagnosis auscultation criterion may be met when the auscultation unit detects rales, crackles, or moist rales. The moist rales refer to auscultation sounds that are similar to sounds of rubbing hair.

Also, the second pneumonia diagnosis auscultation criterion may be met when the auscultation unit fails to detects breaths during a pulmonary region auscultation.

Also, the pneumonia diagnosis body temperature criterion may be met when the body temperature measurement unit detects fever that exceeds a predetermined range.

Reasons for setting the pneumonia diagnosis criteria as above are described below.

In medicine, the common cold (upper respiratory infection) is an acute viral rhinopharyngitis, a highly infectious disease that is caused when the upper respiratory system is affected by viruses. The disease is very common in that even adults are infected many times a year. In particular, infants and young children may be infected 6 to 8 times or more a year, and accordingly, a prevalence rate is very high. Cold may be caused by various viruses. If not severe, cold may be naturally cured by resting without consuming medicine. For infants and young children, in order to alleviate symptoms of cold, the body temperature is reduced by using fever remedies such as acetaminophen or brufen.

Although cold itself is not life threatening, pneumonia, which is a complication of cold, may be life threatening for children, the elderly, and the disabled, and thus is a matter of deep concern. Pneumonia is a main cause of the death of children of age 5 or under worldwide, and the cause of 13% of infectious diseases in age 2 or less children.

Although a thermometer is the most commonly used instrument at homes to observe symptom changes of a cold patient, it is difficult to distinguish cold and pneumonia by only using the thermometer. A combination of the thermometer and a stethoscope may allow relatively more accurate evaluation of a pneumonia infection possibility. For example, the World Health Organization (WHO) defines a respiratory rate for determining child pneumonia as below:
children under 2 months of age: 60 or more rapid breaths per minute
children between 2 to 11 months of age: 50 or more rapid breaths per minute
children between 12 to 59 months of age: 40 or more rapid breaths per minute

Furthermore, pneumonia, which occurs as a complication of cold, may be diagnosed at an early stage by auscultation signs such as crackles and rales.

For diagnosis of pneumonia compilations in a child infected with cold, body temperature measurement as well as evaluation of auscultation sounds using a stethoscope may be used as an instrument for diagnosing an early stage of cold converting into pneumonia. Typical symptoms of child pneumonia include coughs, fever, chest pain, and respiratory symptoms such as rapid breathing.

As described above, in the case of a patient suspected of pneumonia, auscultation sounds and local or systematic fever that occurs according to infection symptoms may be detected at the same time, and thus, an early pneumonia diagnosis rate may be increased.

Also, in the case of pneumonia caused by atelectasis, it is important to check auscultation sounds and fever. Atelectasis refers to a state in which air is absent in the lung and a portion of the lung is as a rubber balloon with no air inside. There are many mild cases with no symptoms, however, hypoxia, difficulty in breathing, fever and chills due to secondary respiratory infection, and chest pain may occur. In particular, severe hypoxia and respiratory failure may lead to death.

Auscultation signs of Atelectasis includes breathing sounds at a certain portion cannot be heard. When an atelectasis infected portion affected by secondary viral infection and transits to viral pneumonia, systematic fever due to pneumonia and auscultation signs of pneumonia are present. Accordingly, a diagnosis possibility may be increased by simultaneously acquiring a body temperature and auscultation signs.

Therefore, the above-described compound medical device may measure the body temperature of the patient by using the body temperature measurement unit, and determine whether fever due to infection is present. Also, when breathing sounds are not detected from a portion where breathing sounds have to be detected by an auscultation unit, the patient may be determined as showing atelectasis symptoms.

Therefore, the compound medical device may determine that the patient is infected by atelectasis and showing fever symptoms due to the infection by using the, body temperature measurement unit and the auscultation unit. In this case, the compound medical device may determine that pneumonia due to atelectasis symptoms has occurred in the patient.

Also, various allergic diseases such as allergic rhinitis and asthma cannot be distinguished from cold and pneumonia in children especially. Thus, treatment timing for chronic allergic diseases are easily missed.

Allergic diseases may generally be distinguished from cold by analyzing auscultation sounds. In particular, allergic diseases may be more accurately distinguished based on facts such as systematic fever rarely occurring in allergic diseases. Therefore, an analysis of auscultation sounds and regular measurement of body temperature may increase a possibility of distinguishing cold and allergic respiratory diseases such as allergic rhinitis and asthma, and thus, diagnosis and treatment may be performed at an early stage.

Although chronic obstructive pulmonary disease (COPD) is a common disease that is the fourth leading cause of death according to the WHO, there is no evident cure. Rapid worsening of COPD is referred to as COPD exacerbation.

This symptom gradually worsens and is a painful phenomenon that occurs at an average of 3 times a year. A typical symptom of COPD is coughs, sputum amount increase, sputum color variation, suffocating feeling in the lungs, auscultation sounds such as wheezing, and fever. The analysis of auscultation sounds and the regular measurement of body temperature variation may increase a possibility of diagnosing and curing rapid worsening of COPD at an early stage.

For example, in the compound medical device, the controller may electrically analyze the auscultation sounds detected by the auscultation unit, and based on waveforms of sound waves, determine whether wheezing is detected. Also, the compound medical device may measure the body temperature of the patient by using the body temperature measurement unit, and when fever exceeding a normal range is detected, diagnose rapid worsening of COPD at an early stage.

Hereinafter, an algorithm for diagnosing enteritis infection by using auscultation sounds and body temperatures in an enteritis auscultation mode will be described.

When the mode of diagnosis is an enteritis mode, the patient may be diagnosed with enteritis by the controller of the compound medical device when a first enteritis diagnosis auscultation criterion and a second enteritis diagnosis auscultation criterion are both satisfied.

The first enteritis diagnosis auscultation criterion may be met when an auscultation unit detects metallic bowel sound. The second enteritis diagnosis auscultation criterion may be met when a heart rate detected by the auscultation unit and a body temperature increase rate detected by the body temperature measurement unit both exceed a statistically normal range.

With regard to this, enteritis may be divided into viral and bacterial enteritis. Enteritis that is common for children are usually viral, and the most well-known is pseudocholera. Symptoms include high fever, vomiting, diarrhea. However, at an early stage of infection, fever occurs. Usually 2 or 3 days of fever occurs with vomiting, and then, vomiting slightly decreases and diarrhea occurs.

Since early stage symptoms of enteritis are similar to common respiratory infection symptoms, diagnosis may be delayed and wrong treatment may be performed. Body temperature measurement and an analysis of intestinal movement auscultation sounds may allow distinguishing of respiratory diseases and enteritis at an early stage, and thus lead to appropriate diagnosis and treatment as well as providing of appropriate treatment opportunities at an early stage.

Typical symptoms of rotaviral enteritis, which is the most common in children, include dehydration, metallic bowel sound due to increase in intestinal movement according to auscultation signs, and a mismatch between a heart rate and a body temperature increase rate.

Accordingly, other than early stage fever, without changes in respiratory auscultation sounds of pneumonia, a diagnosis rate may be increased based on abdomen auscultation sounds due to excessive intestinal movement. Along with cardiac sound auscultation, a mismatch between a heart rate and a body temperature increase rate may be detected, and thus, diagnosis accuracy may be increased.

Irritable bowel syndrome is a disease that involves abdominal pain, bloating, and changes in bowel habits without evident organic disorders. Irritation bowel syndrome is a common chronic disease with a prevalence rate of 10% to 20% and an incidence rate of 1% to 2%.

For irritable bowel syndrome, an occurrence of non-specific symptoms is a warning sign indicating the need to find other organic reasons. Progressive symptoms and systematic fever are examples of non-specific symptoms. Existence of progressive symptoms may be determined by repeating quantitative evaluation of intestinal movement according to an auscultation sound analysis performed by the auscultation unit of the compound medical device.

Also, non-specific symptoms may be detected by performing evaluation of intestinal movement and regular measurement of body temperature variation by using the body temperature measurement unit. Systematic evaluation of Intestinal movement and body temperature variation may distinguish an occurrence of acute diseases such as enteritis.

Hereinafter, an algorithm of diagnosing health status of a pregnant woman and a fetus by using auscultation sounds and body temperatures in a pregnant woman health status auscultation mode will be described.

When the mode of diagnosis is a prenatal checkup mode, the patient may be diagnosed with a normal pregnancy by the controller of the compound medical device when a pregnancy heart rate criterion and a pregnancy body temperature criterion are both satisfied.

The pregnancy heart rate criterion may be met when a fetal heart rate detected by the auscultation unit is within a predetermined heart rate range according to a normal fetal heart rate chart.

The pregnancy body temperature criterion may be met when a body temperature of the patient detected by the body temperature measurement unit is within a predetermined range according to a normal basal body temperature chart.

Related to this, a body temperature and a heart rate of a pregnant woman will be described in detail. The body temperature of the pregnant woman slightly decreases. It is understood that this decrease is a reaction caused by an increase in heat production because of a metabolic rate increase due to pregnancy and a direct heat production of a fetus. In average, the body temperature of the pregnant woman decreases 0.3° C. during the first 3 months and decreases 0.1° C. every month (Wang and Apgar, 1998). With regard to importance of body temperature adjustment, the pregnant woman has to move with caution such that a body temperature increase or dehydration does not occur.

The stethoscope may be used as an instrument for observing the health status of the fetus. Heart pulses of the fetus may be heard through the abdominal walls of the pregnant woman. First beating of the heart starts from the fifth gestational week, and is similar to the beating of the pregnant woman, 80 to 85 beats per minute. During the first month, the heart pulse increases everyday by 3 beats per minute. This variation is very accurate that the variation may be used instead of ultrasound examination when medically calculating the gestational week. At the ninth gestational week, the heart rate of the fetus increases to an average of 175 beats per minute. From this point, the heart rate decreases to a normal rate for a mid-phase of pregnancy, i.e., 120 to 180 beats per minute. The heart rate gradually decreases even during the tenth week.

Although there are reports about harms of an ultrasound unit to a fetus, which is used to listen to cardiac sounds, a stethoscope is safe in that the stethoscope detects generated sounds and thus does not affect the fetus. As described above, the pattern of decreasing in the body temperature of a pregnant woman and the pattern of generation and decreasing in the cardiac sounds of a fetus are shown to be related to each other. Therefore, when the body temperature of the pregnant woman and the cardiac sounds of the fetus are systematically linked and monitored, this would provide a great amount of assistance for an accurate diagnosis of a mother-fetus relationship and a healthy pregnancy process.

For example, the above-described compound medical device may store data about a body temperature decrease pattern of a pregnant woman per gestational week and a table of heart rates of a fetus. When the patient is a pregnant woman, the compound medical device may measure a body temperature of the pregnant woman by using the body temperature measurement unit, and detect a fetal heart rate by using the auscultation unit. The controller of the compound medical device may determine a normal range of the body temperature of the pregnant woman and a normal range of cardiac sounds of the fetus based on a gestational week of the pregnant woman that is input via a user input unit. When the detected body temperature of the pregnant woman and the detected heart rate of the fetus are outside the normal ranges, the compound medical device may determine that the pregnant woman and the fetus have health disorders.

Also, when the compound medical device is used to estimate a gestational week, a process of receiving an input of the number of pregnancy weeks via the user input unit may be omitted. The compound medical device may compare a fetal heart rate detected by the auscultation unit and a body temperature of a pregnant woman detected by the body temperature measurement unit respectively with a body temperature table of a pregnant woman per gestational week and a heart rate table of a fetus per gestational week.

The compound medical device may estimate a gestational week of the pregnant woman by performing a comparison with the body temperature table of the pregnant woman and the heart rate table of the fetus.

A device that includes a body temperature measurement unit, an auscultation unit, and a controller that are manufactured as described above is not limited to medical purposes.

For example, the device that includes the above-described body temperature measurement unit, the above-described auscultation unit, and the above-described controller may function as an educational device or a toy.

For example, when the device is used as an educational device, the device may be used as an educational supplementary instrument for teaching principles of the human body and diagnosis algorithms to an ordinary student that studies or has interest in medicine. For example, the device according to an embodiment of the present specification may be used to a patient or an educational human body model that is manufactured for educational purposes to show symptoms of a suspected disease.

Therefore, a user of the device according to an embodiment of the present invention does not have to be a doctor. A doctor, a medical student, or an ordinary user may all use the device.

Also, a patient of the device according to an embodiment of the present invention does not always have to be human, and may include a human body, a body of an animal, and an educational supplementary material for medical education.

When the device is used as a toy, children may use the device on a patient (or an educational human body model).

The above-described embodiments may be implemented as methods, apparatuses, or articles using standard programming and/or engineering techniques. The term “articles” include computer programs that may be accessed from a random computer-readable apparatus, carriers, or media. For example, computer-readable media includes, but is not limited to, magnetic memory devices (e.g., hard disks, floppy disks, and magnetic strips), optical disks (e.g., CDs, DVDs), smart cards, and flash memory devices (e.g., EEPROMs, cards, sticks, key drives). Also, various types of storage media described herein include at least one device for storing information and/or other machine-readable media.

The term “machine-readable media” includes, but is not limited to wireless channels and various types of other media which may store, hold, and/or transmit command(s) and/or data.

Descriptions of the embodiments above are provided so that one of ordinary skill in the art to which the present invention pertains may user or perform the present invention. Various changes in form and details of the embodiments will be evident to one of ordinary skill in the art to which the present invention pertains, and general principles defined herein may be applied to other embodiments without departing from the scope of the present invention. Therefore, the present invention is not limited to the embodiments provided herein, and should be interpreted in the broadest range that is consistent with the principles and new features provided herein.

### DESCRIPTION OF REFERENCE NUMERALS

| | |
|---|---|
| 1: CASE OF COMPOUND MEDICAL DEVICE | 1a: SOUND ABSORBING CUP |
| 1b: SUPPORTING TUBE | 2: CENTRAL CONTROL MODULE |
| 3: LCD WINDOW | 4: SWITCH BLOCK |
| 5: MODE SWITCH BLOCK | 6: SPEAKER |
| 7: ANTENNA | |
| M: MICROPHONE | T: TEMPERATURE SENSOR |
| V: POWER SUPPLY | |

2-1: AUSCULTATION MODE DESIGNATION UNIT

| | |
|---|---|
| 2-2: DETECTOR | 2-3: FILTER |

2-4: AUSCULTATION SIGNAL AMPLIFIER

| | |
|---|---|
| 2-5: A/D CONVERTER | 2-6: DIGITAL SIGNAL AMPLIFIER |
| 2-7: DATABASE | 2-8: COMPARING UNIT |
| 2-9: CONTROLLER | 2-10: STORAGE |
| 2-11: OUTPUT UNIT | 2-12: DISPLAY |
| 2-13: AUDIO UNIT | 2-14: TRANSCEIVER |

2-15: BODY TEMPERATURE MEASUREMENT UNIT

1. A compound medical device comprising:
an auscultation unit receiving sounds inside a body of a patient;
a body temperature measurement unit measuring a body temperature of a contact portion;
a controller connected to the auscultation unit and the body temperature measurement unit, and determining health status of the patient based on an input received from the auscultation unit and the body temperature measurement unit; and
an output unit outputting the health status of a patient determined by the controller.

2. The compound medical device of claim 1, further comprising a body data input unit receiving at least one of age, sex, height, weight, gestational week of the patient, and a mode of diagnosis of the patient from a user.

3. The compound medical device of claim 2, wherein when the mode of diagnosis is a pneumonia mode, the patient is diagnosed with pneumonia by the controller diagnoses that the patient is infected with pneumonia when both of a pneumonia diagnosis body temperature criterion and at least one of pneumonia diagnosis auscultation criteria are satisfied,
the pneumonia diagnosis auscultation criteria comprise a first pneumonia diagnosis auscultation criterion, a second pneumonia diagnosis auscultation criterion, and a third pneumonia diagnosis auscultation criterion,
the first pneumonia diagnosis auscultation criterion is met when the auscultation unit detects rales, crackles, or moist rales,
the second pneumonia diagnosis auscultation criterion is met when the auscultation unit fails to detect a respiration rate during a pulmonary region auscultation, and
the pneumonia diagnosis body temperature criterion is met when the body temperature measurement unit detects body temperature changes that exceeds a predetermined range.

4. The compound medical device of claim 3, wherein the pneumonia diagnosis auscultation criteria further comprises a pediatric pneumonia diagnosis criterion,
when the pediatric pneumonia diagnosis criterion is satisfied, the patient is diagnosed with pneumonia by the controller, and
the pediatric pneumonia diagnosis criterion is met when the auscultation unit detects 60 or more breaths per minute when the patient is a child under 2 months old, 50 or more breaths per minute when the patient is a child between 2 to 11 months old, or 40 or more breaths per minute when the patient is a child between 11 to 59 months old.

5. The compound medical device of claim 2, wherein when the mode of diagnosis is an enteritis mode, the patient is diagnosed with enteritis by the controller when a first enteritis diagnosis auscultation criterion and a second enteritis diagnosis auscultation criterion are met,
the first enteritis diagnosis auscultation criterion is met when the auscultation unit detects metallic bowel sound, and
the second enteritis diagnosis auscultation criterion is met when a heart rate detected by the auscultation unit exceeds a statistically normal range and a body temperature increase rate detected by the body temperature measurement unit exceeds a statistically normal range.

6. The compound medical device of claim 2, wherein when the mode of diagnosis is a prenatal checkup mode, the patient is diagnosed with a normal pregnancy when both of a pregnancy heart rate criterion and a pregnancy body temperature criterion are met,
the pregnancy heart rate criterion is met when a fetal heart rate detected by the auscultation unit is within a predetermined range according to a normal fetal heart rate chart, and
the pregnancy body temperature criterion is met when a body temperature of the patient detected by the body temperature measurement unit is within a predetermined range according to a normal basal body temperature chart.

7. The compound medical device of claim 1, further comprising a transceiver transmitting body temperature information detected by the body temperature measurement unit and auscultation sounds detected by the auscultation unit, and
wherein the auscultation sounds comprise at least one of blood flow sounds, breathing sounds, and bowel sounds.

8. The compound medical device of claim 1, wherein the auscultation unit comprises:
an ultrasound transmitter emitting ultrasound waves into the body of the patient; and
an ultrasound receiver detecting echo waveforms of the ultrasound waves that are emitted into the body of the patient.

9. The compound medical device of claim 1, further comprising an otoscope,
wherein the otoscope comprises a lighting unit that illuminates an inside of the body of the patient and a magnifying lens.
